(19) United States
(12) Patent Application Publication
(10) Pub. No.: US 2025/0140409 A1
(43) Pub. Date: May 1, 2025

(54) ARTIFICIAL INTELLIGENCE AND BIOINFORMATICS SYSTEM FOR AGE-RELATED MACULAR DEGENERATION

(71) Applicant: iMacular Regeneration LLC, Rochester, MN (US)

(72) Inventor: Timothy W. Olsen, Rochester, MN (US)

(21) Appl. No.: 18/914,736

(22) Filed: Oct. 14, 2024

Related U.S. Application Data
(60) Provisional application No. 63/546,063, filed on Oct. 27, 2023.

(51) Int. Cl. G16H 50/20; A61B 3/12; G16H 10/40; G16H 30/40; G16H 50/70

(52) U.S. Cl. CPC G16H 50/20; A61B 3/12; G16H 10/40; G16H 30/40; G16H 50/70

(57) ABSTRACT

Methods and systems of data analysis in connection with age related macular degeneration (AMD) are described. Among other examples, techniques are described for generating a bioinformatics database of AMD data, based on correlation of live patient data with eye bank (donor) patient data. Such techniques may include performing data analysis to correlate characteristics of live patient image data with characteristics of eye bank image data, with the correlated characteristics including common image data characteristics of each set of image data based on a disease progression of AMD. Further data analysis may correlate tissue data characteristics from eye bank donor eyes with the common image data characteristics based on the disease progression of AMD. Still further data analysis may correlate clinical data characteristics from live patient observation data with eye bank observation data and with the common image data characteristics based on the disease progression of AMD.

This application claims the benefit of priority to U.S. Provisional Patent Application Ser. No. 63/546,063, filed Oct. 27, 2023, which is incorporated herein by reference in its entirety.

### TECHNICAL FIELD

Embodiments described herein generally relate to medical data analysis, and particularly to data analysis performed in connection with image, clinical, biologic, and tissue data associated with ophthalmologic diseases.

### BACKGROUND

Age-related macular degeneration (AMD) is the leading cause of irreversible blindness in those over age 50 in the Western world (Beaver Dam Eye Study, 1992). Similar prevalence rates of blindness are reported from East Asia, the world's most densely populated area (Wong, Lancet 2014). Advanced AMD profoundly affects an individual's quality of life. Studies have shown that the quality of life for someone with advanced AMD (visual acuity <20/200 in the better eye or legally blind) is equivalent to being bedridden, having a catastrophic stroke, or advanced prostate cancer (Brown 2006). Affected individuals lose the independence of driving, cannot read, recognize friends, or see their grandchildren's faces. They isolate, withdraw from society, and become depressed. With the number of individuals in the >60-year-old age group increasing, current predictions indicate that the AMD “at-risk” population will be over 350M globally by 2030.

Finding or discovering new therapies and treatments for those suffering from AMD or who are at high-risk for progression has developed into an important area of research. However, there are no animal models to study AMD. Rodents (e.g., rats and mice) do not have a macula, and primates with a macula do not live long enough to develop AMD. Although various research efforts have been performed and are ongoing to evaluate different aspects of AMD, research in this area is complicated because AMD is a complex disease and is not caused by a single gene, a single protein, or a single biologic pathway.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 depicts a color fundus photographic image of the posterior segment of a human eye as an illustrative example of live patient data.

FIG. 2 depicts a color fundus photographic image of the posterior segment of a human donor eye as an illustrative example of eye bank data.

FIG. 3 depicts a schematic of a data processing system.

FIG. 4 illustrates an example method for generating a bioinformatics database of AMD data.

FIG. 5 illustrates a diagram of artificial intelligence (AI) engines for training and execution, usable to perform data processing operations according to the present disclosure.

FIG. 6 illustrates a block diagram schematic of various components of an example machine that may be used as, for example, a computing device, cloud service, or a server, usable to perform computing operations according to the present disclosure.

### DETAILED DESCRIPTION

The present disclosure generally relates to data collection and processing operations related to many aspects of AMD. These data processing operations can be used to identify and recommend effective therapeutic targets and therapies to treat AMD, based on correlations and findings produced from advanced data analysis.

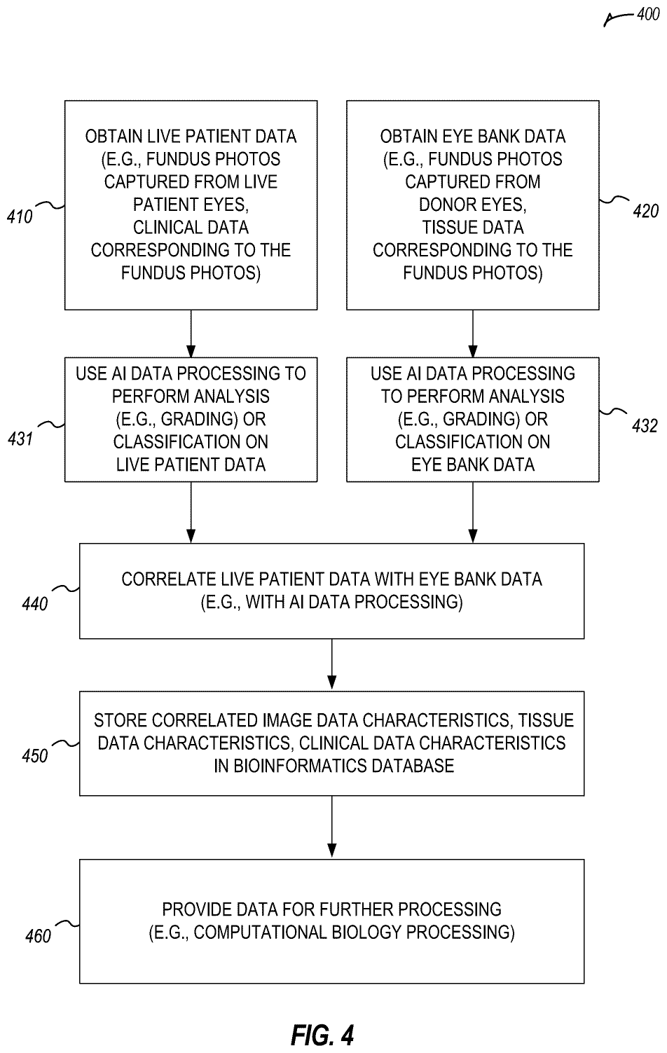

In particular, the following discusses various aspects of artificial intelligence (AI) and bioinformatics data processing on multiple sources of data observations for AMD, including the analysis and correlation of live and donor data (including, with the merging of data processing pathways for this data, discussed with reference to FIG. 4, below). The live patient data observations may be provided from research projects (e.g., Age-Related Eye Disease Study (AREDS) and the Intelligent Research in Sight (IRIS) registry) that have collected data from many unique patients and patient disease states. These live patient data observations correspond to detailed data provided from human eye banks (e.g., collections of postmortem donor eyes). Eye bank data includes, but is not limited to, detailed fundus imaging and tissue samples taken directly from donor eyes that have been processed. Such fundus images, when graded or ranked (e.g., with the Minnesota Grading System or “MGS”), can indicate a precise state of macular degeneration progression that can be correlated to other biological characteristics found in tissue. Tissue samples are analyzed using a variety of biologic analysis techniques (BATs) at pre-determined stages of AMD that includes, but is not limited to, proteomic (quantitative and qualitative), genomic, gene expression, transcriptomic, RNA or DNA sequencing, RNA or DNA methylation, epigenetic modifications, post-translational proteomic modifications, metabolomic, structural biologic, pathway and other analyses.

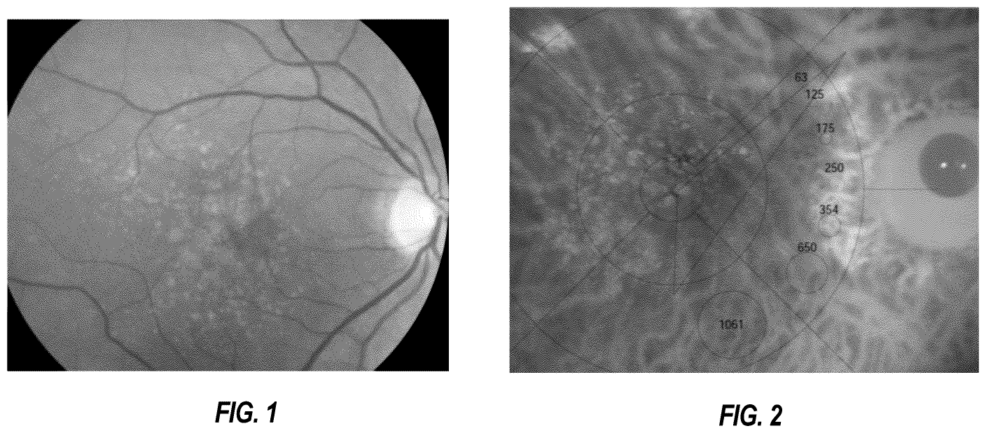

The following also describes uses of AI generated data processing to enable the live patient data from prior AMD studies of live patients (e.g., AREDS data, including imaging and medical record data captured from live patient eyes) to correlate with the eye bank data from donor patients (e.g., data from postmortem donor eyes that are graded according to the multi-step MGS, including imaging, tissue, and medical record data). The relationship is based upon the common phenotype, demonstrated in matching color fundus photographic images of a live human (e.g., depicted in FIG. 1) with color fundus photographic images of a donor eye (e.g., depicted in FIG. 2). Phenotype matching supports the tightly corresponding link between the clinical AREDS database and the eye bank database BATs. Published proteomic data also supports the relationship between phenotype, AREDS data, and a specific BAT.

The correlated data is stored in a bioinformatics database that uses unique phenotypic criteria (both AREDS and MGS-graded eye bank data) based on published models to allow data mining and analysis. The correlated data can be used to represent and compare the exact state and risk of AMD progression for respective phenotypic characteristics appearing from analysis of color fundus photographs taken from an eye, based on specific clinical features identified in the fundus imaging (such as drusen size, shape, quantity, pigmentary changes, atrophy, drusen subtype, and others) and tissue samples (e.g., BATs). This correlated data can be used to create an advanced, computational model to study pathways involved in the pathogenesis of AMD, and the data can be queried or used for additional bioinformatic data processing operations. An AREDS data analysis system (e.g., DeepSeeNet, as described in Ophthalmology 2019 Apr;126 (4): 556-575) that provides an automated grading of images from live, human subjects could be trained to apply automated analysis and grading of images of human donor tissue (e.g., eye bank data to be graded according to the MGS).

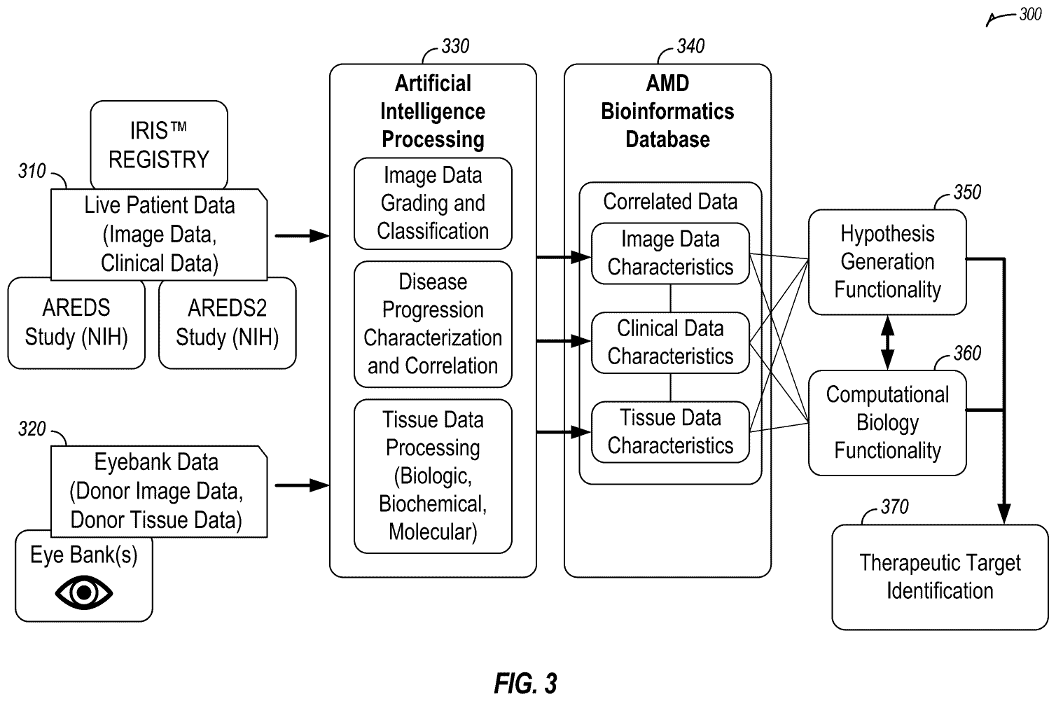

As shown in detail in FIG. 3 and discussed in more detail below, live patient and eye bank donor data is used to generate a layered, bioinformatic database that links a variety of data characteristics to phenotypes (e.g., photos from live patient data such as AREDS and IRIS registry to human eye bank data). The AMD disease phenotype, based on color fundus photos, maintains scientific consistency between AREDS and IRIS registry data to MGS data and identifies a specific disease stage. Phenotype data from photo imaging is the most predictive factor of AMD disease risk-even more predictive than genetic testing-and thus can be used as a primary mechanism to correlate a disease progression state between live patient data and eye bank data.

This layered, bioinformatic database can provide a high-quality source of AMD bioinformatics data, which links to the stages of human disease using a variety of characteristics of image data, clinical data, and tissue data. The correlated data in this bioinformatic database may be further optimized or evaluated with additional artificial intelligence functions and systems biologic approaches to allow for a novel disease modeling of AMD, thereby generating data for use by subsequent medical data processing functions. For example, the bioinformatics database may host data to be analyzed using computational biologic software, and this data may be mined to identify relevant biologic pathways in the various disease stages, molecules involved at various stages, and overall mechanisms or biologic pathways involved in the specific AMD disease progression. Accordingly, the information in the bioinformatic database may be used by various data processing functions to assist in the identification of AMD therapeutic targets, such as by identifying specific molecules, proteins, genes, or pathways that may be expected to be responsive to interventional AMD treatments or therapeutics.

FIG. 1 depicts an image of an eye as an illustrative example of live patient data. This image is a clinical color fundus photograph of a posterior segment of a right eye (back, inside layer of the eye) from a 70-year-old. The image shows the optic nerve (yellow circle on right), retinal blood vessels (red tree-branch structures), and soft, yellow spots in the center (macula) that represent drusen. In this clinical fundus photo, there are many yellow drusen which are a strong indicator of risk for progression to advanced age-related macular degeneration. In fact, this imaged eye has a 50% chance of progression to vision loss or legal blindness within the next 5 years (based on outcomes of prospectively studied subjects in the AREDS). The current visual acuity is measured at 20/20 (normal vision).

The prospective data (public database) provided from the AREDS, AREDS2, or similar long-term patient studies provides information on the natural disease history and disease progression. Multiplying the total number of patients by the number of years followed in both AREDS/AREDS2 studies results in 36,000 prospectively studied people-years. AREDS and AREDS2 looked at individuals prospectively and collected numerous annual color fundus photos, similar to the eye image depicted in FIG. 1. Among other findings, AREDS found that drusen (yellow spots) and pigmentary changes visible in color fundus photos represent the best predictors of AMD progression. The simplified AREDS grading system used a four-step, progressive classification to predict progression to end-stage AMD. Later analysis of the AREDS data defined a nine-step progressive classification scale that was capable of more accurately predicting the risk of progression to end-stage AMD. Thus, the annual color fundus photos captured by AREDS identify an extensive data set available to document the natural history of AMD progression.

FIG. 2 depicts an image of an eye as an illustrative example of eye bank data. This image is a color fundus photograph of a posterior segment of a right eye from a deceased 80-year-old who donated their eyes to an eye bank. The back of the eye was imaged and classified using methods defined by the MGS, which is based on identical phenotypic definitions as AREDS/AREDS2. MGS provides a model system to study AMD from affected human donor eyes, based on specific photographic features, using similar photographic techniques (stereo color fundus photographs) and grading templates. The grading scale used by MGS, in both the MGS-4 and MGS-9, uses the exact grading criteria from AREDS/AREDS2 and has been published as either the MGS-4 (Invest Ophthalmol Vis Sci. 2004;45;12;4484-4490) or MGS-9 (Invest Ophthalmol Vis Sci. 2017;58:5497-5506). Prior to the AREDS grading system, the phenotype criteria were based on, and slightly modified from, the Wisconsin Age-Related Maculopathy Grading System (WARMGS). With either grading scale, MGS provides the ability to capture consistent stereoscopic, color fundus photographs of the macula in postmortem eye bank eyes to correspond to the AREDS/AREDS2 photographic database.

The use of the MGS grading scale may include many additional phenotypic features that modify the risk classification from information found in the fundus image. Examples of these features include, but are not limited to drusen subtype, drusen distribution, pigmentary changes, pigment detachments, the presence of blood or fibrosis in various patterns and configurations, blood vessel abnormalities, and others. In the image depicted in FIG. 2, there is a red sphere added into the photograph and placed directly on the optic nerve. This is a 1000-micron ruby sphere that is used as a size reference for the overlying template grid along with the associated size demarcated circles. The grid and size circles allow for precise quantification of yellow spots or drusen to correspond exactly to the macular degeneration stages defined by AREDS, which can be used to create data records including measurements of respective characteristics of the postmortem eyes.

Additionally, in this fundus image (in FIG. 2), the retinal blood vessels have been removed. Removal of the retina is needed to photograph the drusen in donor eyes, because the retina becomes opaque post-mortem. While the techniques for fundus photography differ, a living subject's retina is transparent (and is invisible to photography, except for the retinal blood vessels). Additionally, when applying the MGS to donor eyes, the blood vessels are imaged prior to removing the retina. Therefore, removing the retina from the donor eyes only generates a negligible difference from fundus images taken from a living subject.

Detailed BATs may be obtained directly from donor eye tissue(s) and may be analyzed soon after the donor eye has been imaged and graded. The BATs from the graded tissue can be studied using many of the multi-omic, bioinformatic technologies. A significant advantage of using donor eyes and applying a consistent grading scheme for donor eyes (e.g., MGS) is that a grading scheme enables the study of early stages of AMD directly from human eye tissue. Thus, correlating BAT data at the early “at-risk” disease stage will help determine the relevant pathways in the progression of AMD progression and identify potential target pathways for treatment. These disease progression patterns are based on the prospective data from AREDS and applied to donor eyes (graded with the MGS) that have the specific AMD disease phenotypic correlates.

The following describes a data processing system configured to use and compare various AI processing operations that evaluate live patient data (including but not limited to human or AI graded, AMD fundus photos) and eye bank donor data (including but not limited to photographed donor eyes, linked to associated biology, histology, genetics, and multi-omics data from tissue samples). In an example, AI processing functions are used to perform a direct comparison and correlation of characteristics of a live patient eye image with AMD, to a donor eye image at the same AMD stage. This produces correlated data that will associate (link) imaging attributes with clinical attributes and with tissue attributes, all at the same stage of AMD progression.

An important benefit of the proposed analysis is that additional information is provided from a donor eye-image data and tissue-derived BATs data-that is not available from living patients because obtaining a biopsy for tissue cannot be performed on a living human's eye. Therefore, this analysis represents the basis for a tremendously powerful research tool to study and discover new relevant biologic pathways involved in and therapeutic targets for those afflicted by various stages of AMD as well as overall AMD pathogenesis. Consequently, this system may be contrasted with existing approaches for in-vivo classification and review of AMD disease states, including trained AI models that predict a disease progression from a live patient image that is based on training of previous outcomes from other live patient images. As will be understood, the exclusive use of images and clinical data from live patients will not be able to provide the BATs data or other detailed biochemical, genetic, and histologic attributes of AMD disease that are obtainable only from analysis of donor eyes.

FIG. 3 depicts a schematic of a data processing system 300 according to the present examples. At a high level, this data processing system 300 may be used to create and maintain an AMD bioinformatics database 340 that is populated with correlated data. This AMD bioinformatics database 340, once populated, may be mined using additional software functions (e.g., hypothesis generation functionality 350, computational biology functionality 360) to answer specific, hypothesis-generated inquiries. This AMD bioinformatics database 340 may be populated from functions of AI processing 330, including the results of specific data processing operations (e.g., the use of AI techniques to correlate disease progression between multiple sets of source data, or the use of AI techniques to identify, classify, define, and correlate characteristics of the source data).

In an example, the primary data entry sources for the data processing system 300 includes eye bank data 320 comprising donor image data and associated donor tissue data, and live patient data 310 comprising image data and associated clinical data from AREDS and additional clinical data from the IRIS registry. The images in the eye bank data 320 may be graded using fully automated techniques performed by the AI processing 330, or may be previously graded (e.g., manually graded using MGS or another grading approach) and then validated using the AI processing 330. Accordingly, the eye bank data 320 may include previously graded or ungraded image information represented in a variety of formats.

In a specific example, machine learning techniques are used to automate grading of color fundus images from the eye bank data 320, such as the use of a trained model to apply a classification to respective stereoscopic color fundus images according to the MGS-9 grading scale. One or more AI models (implemented by AI processing 330) may be trained and validated to transform the manual grading of tissue into an automated grading process. Once trained and validated, the AI processing 330 may operate a high-efficiency grading protocol for multiple sources and types of source eye bank images. Because current manual grading of MGS tissue is time-consuming and expensive, the use of automation can help achieve a consistent and valid data entry into the AMD bioinformatics database 340.

Graded images from the eye bank data 320 provide the basis for correlation with graded live patient images and clinical information from the live patient data 310, to be stored in the AMD bioinformatics database 340. The AMD bioinformatics database 340 may be structured to associate color fundus images from the AREDS (at some AMD stage) with color fundus images from the eye bank data (at the same AMD stage), and additionally to associate clinical data that is encountered from patients (also at the same AMD stage) and to associate tissue data from the eye bank data (also at the same AMD stage). Data from pre-mortem patient records (i.e., smoking history, diet, age, race, other eye disease, exercise history, lifestyle, etc.) can be imported when medical records are available from the donor (associated with eye bank data 320).

AI processing 330 may be used to identify and import other aspects of live patient data 310, including characteristics of images and clinical data relevant to AMD. In addition to images, AREDS provides high quality clinical, dietary, nutritional, and lifestyle information related to AMD progression. However, eye tissue (and associated biochemistry, proteomics, etc.) is not available to study directly from patients in AREDS. Access of the AREDS and IRIS registry data is coordinated to identify relevant data characteristics to be imported into the AMD bioinformatics database 340.

Additional “Big Data” input from other clinical data sources can be layered in the AMD bioinformatics database 340, including data from AREDS scientific data measurements and publicly available data studies related to AREDS (e.g., from over 70 publications that have provided AREDS/AREDS2 data findings). Thus, data related to the IRIS and AREDS studies may be jointly mined or extracted by the AI processing 330 to identify common clinical characteristics or attributes of a precise stage of AMD disease.

In an example, the AMD bioinformatics database 340 may represent correlated data using one or more unique data structures that are linked by a risk-stratified AMD phenotype. Fundus photographs (e.g., shown in FIGS. 1 and 2) represent an example of the most precise AMD disease-staging analysis. Epidemiologic, clinical (e.g., AREDS and IRIS) and grading (e.g., MGS) data are all risk-stratified using common definitions. By correlating these data types, the AMD bioinformatics database 340 may provide a digital, data-intensive, human model of AMD. In particular, the use of a grading system such as MGS enables a precise clinical definition to be applied to human eye bank tissue and allows disease mechanisms present in the tissue to be reviewed in more detail.

The correlated data maintained in the AMD bioinformatics database 340 may be verified or reviewed, including via automated or manual processes such as the additional uses of AI processing 330. The data review may ensure that input data is reliable, validated, and searchable. This may involve aspects of clinical knowledge and data-science expertise that tune data representations maintained in the AMD bioinformatics database 340, or that tune processing operations performed by the AI processing 330. For example, domain expertise from an AMD expert may provide ground truth references for training an AI model or modifying the AREDS DeepSeeNet, and a bioinformatics expert may validate that relevant and appropriate data is included and appropriately labelled during the construction stages of the AMD bioinformatics database 340. Aspects of this validation may also be automated within the system 300.

Additionally, errors of the data processing can be avoided with supervised or semi-supervised (for larger datasets) learning with a human interface to ensure accuracy of the AI machine-learning process. For example, when importing image data, ground-truths (including grading criteria from an expert) can be used to appropriately label data. In an example, all MGS-scored image input into the AMD bioinformatics database 340 uses a supervised learning phase to ensure high quality interpretation. Because the image data considered by MGS may have up to millions of data points, the AI processing 330 may learn data correlations or patterns that are not readily visible or apparent on visual inspection of the images. Accordingly, additional features or rules may be used to confirm data results, including to validate primary data outputs by supervising machine learning inputs into the AMD bioinformatics database 340 (and to correct errors in output, especially false positives).

The data set provided in the AMD bioinformatics database 340 can be validated, risk-stratified, used to provide a specific classification of attributes of human tissue. This provides a source of data that can represent the true clinical disease state for additional molecular studies. For example, the inquiries of the AMD bioinformatics database 340 (e.g., data mining) may originate from hypothesis generation (e.g., coordinated by hypothesis generation functionality 350), to allow data to be interrogated using sophisticated computational biology software (e.g., computational biology functionality 360). Proteomic, genomic, and other molecular data may be directly added into or refined within the AMD bioinformatics database 340 to be accessible by these functions, or accessible via other systems-biology databases and data sources.

As non-limiting examples (e.g., BATs, as described above), the combination of proteomic, genomic, and transcriptomic data using-omic software platforms, integrated with the system 300, can be used to detect novel disease mechanisms (such as mitochondrial pathways using MGS graded donor tissue, or other relevant genomic data). Approaches may be validated with bioinformatic software to discover one of many pathways in the pathogenesis of AMD using human MGS tissue. Findings directly derived from this tissue-which cannot be obtained from live human patients-can be used for determining future pharmacotherapies via identification of molecular pathways. As will be understood, the establishment of the AMD bioinformatics database 340 can support a large number of research scenarios used for identifying therapeutic targets and evaluating the effects of treatments relative to an AMD disease stage.

FIG. 4 illustrates a flowchart of an example method for generating (e.g., manufacturing, making, establishing, updating, etc.) a bioinformatics database of AMD data. Consistent with the examples above, this method may be computer-implemented, performed by a computer system, or embodied by software instructions stored on computer-readable medium.

At 410, the flowchart 400 includes obtaining (e.g., retrieving, accessing, extracting, etc.) a first set of data (live patient data), such as a set (one or more) of color fundus photo(s) captured from live patient eyes and clinical data corresponding to the fundus photo(s).

As used herein, live patient data may include separate or combined data sets comprising live patient image data (e.g., one or more color fundus photos captured from live patient eye(s)) and live patient observation data (e.g., clinical data). Live patient image data may include the use of multiple images of the same eye (e.g., stereoscopic color fundus images of the same eye), multiple images of different eyes, multiple images of different eyes from the same or multiple patients, etc. At 431, the flowchart 400 includes the use of data processing operations (including, as applicable, AI-based image operations) to perform analysis or classification on the live patient data, such as to identify relevant data types and values for additional processing and correlation with eye bank data (discussed below). As suggested above, such live patient data may include data measurements directly captured from live patient studies or observations, or from data values extracted or derived from published data sets or large data studies of live patients (e.g., AREDS, AREDS2, IRIS registry data, etc.).

At 420, the flowchart 400 includes obtaining (e.g., retrieving, accessing, extracting, etc.) a second set of data (eye bank data), such as a set (one or more) of color fundus photo(s) captured from donor eyes and tissue data corresponding to the fundus photo(s). As used herein, eye bank data may include separate or combined data sets comprising eye bank image data (e.g., color fundus photos captured from postmortem eye(s)) and eye bank observation data (e.g., clinical data). Eye bank image data may include the use of multiple images of the same eye (e.g., stereoscopic color fundus images of the same eye), multiple images of different eyes from the same or different donors, etc. At 432, the flowchart 400 includes the use of data processing operations (including, as applicable, AI-based image classification) to perform analysis (e.g., grading) or classification on the eye bank data.

At 440, the flowchart 400 includes performing data analysis to produce a correlation of the live patient data with the eye bank data, as produced by any of the data processing methods or techniques discussed herein. The correlation operations may also be performed with the use of one or multiple AI models or processing functions. This correlation may include correlating a single type or multiple types of characteristics of the live patient data with characteristics of the eye bank data.

As an illustrative example, characteristics represented in live patient image data (e.g., a first set of fundus photos captured from live patient eyes) can be correlated with characteristics represented in eye bank image data (e.g., a second set of fundus photos captured from postmortem eyes). The correlated characteristics may include common image data characteristics of each image data set based on a disease progression of AMD.

In a further example, characteristics represented in the eye bank data may include tissue data, such as tissue data characteristics produced from biological analysis techniques (BATs) performed on tissue samples from the postmortem eyes. Additional data analysis may be performed to correlate the tissue data characteristics with the common image data characteristics based on the disease progression of AMD, and then store the tissue data characteristics in the bioinformatics database. These tissue data characteristics may be stratified by disease severity of AMD.

In a further example, characteristics represented in the live patient data may include live patient observation data, such as attributes of respective human subjects (patients) whose eye(s) were imaged to produce the images in the live patient image data. Such attributes may be directly or indirectly provided by public databases and data sources (e.g., AREDS, AREDS2, etc.). Characteristics represented in the eye bank data may include eye bank observation data, such as attributes of respective human subjects (donors) who provided donor eye(s) that were imaged to produce the images in the eye bank image data. Additional data analysis may be performed to correlate the clinical data characteristics from the live patient observation data and the eye bank observation data with the common image data characteristics based on the disease progression of AMD, and then store the clinical data characteristics in the bioinformatics database. These clinical data characteristics also may be stratified by disease severity of AMD.

At 450, the flowchart 400 includes storing the correlated image data characteristics, tissue data characteristics, and clinical data characteristics, in a bioinformatics database. This may include storing additional or new attributes, characteristics, and data values that were not previously labeled or classified in the live patient data or the eye bank data.

At 460, the flowchart 400 includes retrieving data from the bioinformatics database, and providing the data for further processing (e.g., with computational biology data analysis). One example of further processing may include using the bioinformatics database to identify a therapeutic target in the tissue data, such as where identification of the therapeutic target is performed using one or more of: proteomic data analysis, transcriptomic data analysis, genomic data analysis, gene expression profiling, RNA or DNA sequencing, RNA or DNA methylation analysis, epigenetic modification analysis, post-translational proteomic modifications, metabolomic biomarker identification, structural biological identification, or therapeutic targeting signaling analysis. Another example of further processing may include providing the common image data characteristics for use in therapeutic target identification, with the therapeutic target identification to be performed by one or more hypothesis generation function or computational biology function implemented in a computing system.

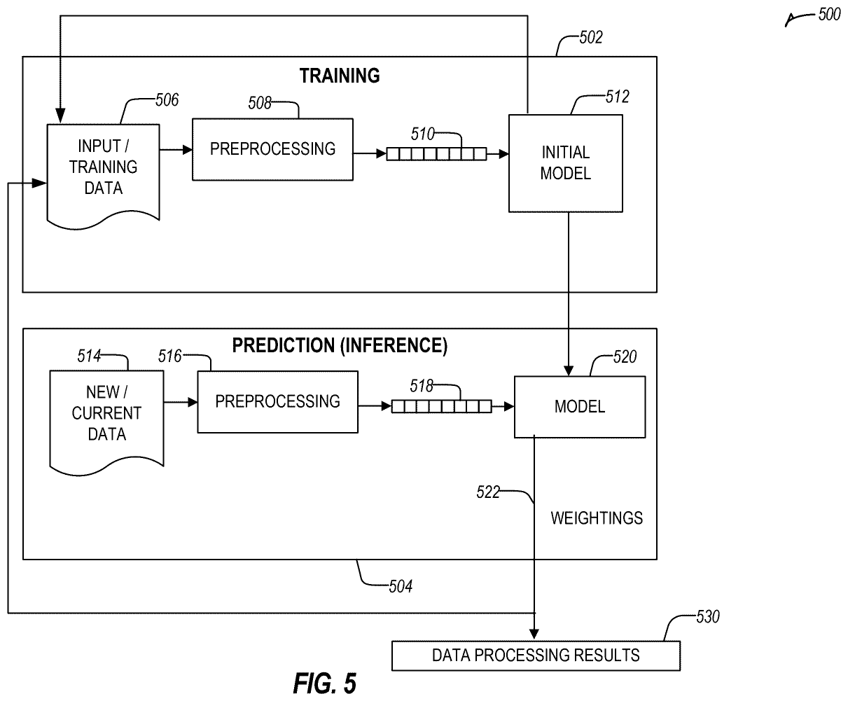

FIG. 5 illustrates artificial intelligence (AI) engines 500 for training and execution, such as may be used for a variety of the data processing operations (e.g., classification, correlation) on live patient data and eye bank data as discussed herein. As shown, a training engine 502, used to perform training operations on a training data set, may be deployed in connection with a prediction engine 504 on a new data set. The engines 500 may utilize a variety of algorithms (e.g., machine learning algorithms) to determine weightings that change the operation and output of the models. In this depiction of training and execution, a variety of data flows, weightings, and algorithms are not shown for simplicity.

As an example configuration, the training engine 502 uses input (training) data 506, such as data provided after undergoing preprocessing component 508, to determine one or more features 510. The one or more features 510 may be used to generate an initial model 512, which may be updated iteratively or with future labeled or unlabeled data (e.g., during reinforcement learning), including to improve the performance of the prediction engine 504 or the initial model 512. An improved model may be redeployed for use. The input data 506 for training may include labeled or unlabeled data, provided in connection with eye bank data, live patient data, or some combination thereof. The input data 506 may further be processed to identify specific image data characteristics, clinical data characteristics, or tissue data characteristics, to train a model, consistent with the examples herein.

In the prediction engine 504, new or current data 514 (e.g., a novel data set) may be input to preprocessing component 516. In some examples, preprocessing component 516 and preprocessing component 508 are the same. The prediction engine 504 produces a feature vector 518 from the preprocessed current data, which is input into the model 520 to generate one or more criteria weightings 522. The criteria weightings 522 may be used to output a prediction or classification (including, to identify common data values or data sets to be correlated between live patient data and eye bank data).

The training engine 502 may operate in an offline manner to train the model 520 (e.g., on a server). The prediction engine 504 may be designed to operate in an online manner (e.g., in real-time at a computer system or server, etc.). In some examples, the model 520 may be periodically updated via additional training (e.g., via updated input data 506 or based on labeled or unlabeled data output in the weightings 522) or based on identified future data, such as by using reinforcement learning to customize a general model (e.g., the initial model 512) to a specific use case or action. The initial model 512 may be updated using further input data 506 until a satisfactory model 520 is generated. The model 520 generation may be stopped according to a specified criteria (e.g., after sufficient input data is used, such as 1,000, 10,000, 100,000 data points, etc.) or when data converges (e.g., similar inputs produce similar outputs).

The specific algorithm used for the training engine 502 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C9.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. A reinforcement learning model may use Q-Learning, a deep Q network, a Monte Carlo technique including policy evaluation and policy improvement, a State-Action-Reward-State-Action (SARSA), a Deep Deterministic Policy Gradient (DDPG), or the like. In other scenarios, examples of artificial neural networks may include perceptron networks, feed forward networks, radial basis networks, deep feed forward networks, recurrent neural networks, Long Short-Term Memory (LSTM) networks, gated recurrent unit networks, auto encoder (AE) networks, variational AE, denoising AE, or sparse AE networks, Markov chain networks, Hopfield networks, Boltzmann machine (BM) networks, restricted BM networks, deep belief networks, convolutional neural networks, deep convolutional neural networks, generative adversarial networks, liquid state machine networks, extreme learning machine networks, echo state networks, deep residual networks, Kohonen networks, support vector machine networks, neural Turing machine networks, and the like. In other examples, unsupervised models may not have a training engine 502. As a non-limiting example, a regression model may be used, where the model 520 is a vector of coefficients corresponding to a learned importance for each of the features in the vector of features 510, 518.

Once trained, the model 520 may output data processing results 530 that are used to provide or identify data for the bioinformatics database discussed above. As an example, the model 520 may consider joint characteristics of live patient data and eye bank data to determine a correlation of specific properties, data values, or conditions. The model 520 may integrate or be supplemented with an additional algorithm or data processing flows that provide relevant information of the data processing results 530. Accordingly, the data processing results 530 from the model 520 and other AI data processing actions may take a variety of forms.

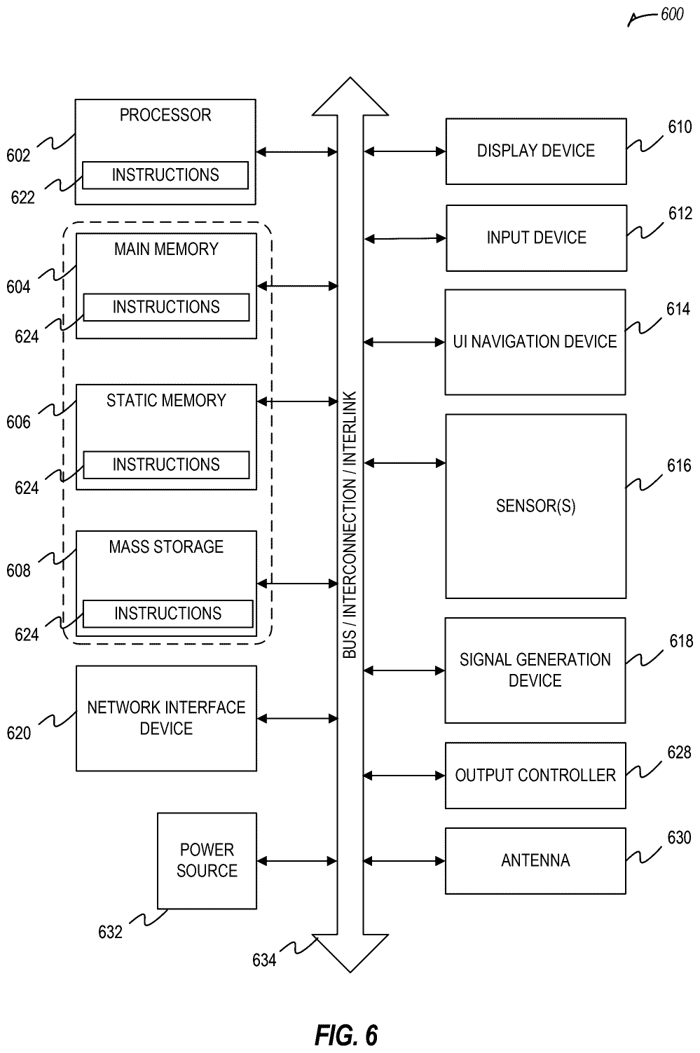

FIG. 6 illustrates a block diagram schematic of various example components of an example machine 600 that can be used as, for example, any of the platforms or systems to implement the AI processing 330, the database 340, the functionality 350, 360, or related hardware devices (e.g., cloud services, server(s), etc.). Examples, as described herein, can generally include, or can operate by, logic or a number of components, modules, or mechanisms in machine 600. Modules may be hardware, software, or firmware communicatively coupled to one or more processors in order to carry out the operations described herein. Generally, circuitry (e.g., processing circuitry) of example machine 600 may include a collection of circuits implemented in tangible entities of the machine 600 that include hardware (e.g., simple circuits, gates, logic, etc.). Circuitry membership can be flexible over time. Circuitries include members that can, alone or in combination, perform specified operations when operating. In some examples, hardware of the circuitry can be immutably designed to carry out a specific operation (e.g., hardwired). In some examples, the hardware of the circuitry can include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions permit embedded hardware (e.g., the execution units or a loading mechanism) to create members of the circuitry in hardware via the variable connections to carry out portions of the specific operation when in operation. Accordingly, in some examples, the machine readable medium elements are part of the circuitry or are communicatively coupled to the other components of the circuitry when the device is operating. In some examples, any of the physical components can be used in more than one member of more than one circuitry. For example, under operation, execution units can be used in a first circuit of a first circuitry at one point in time and reused by a second circuit in the first circuitry, or by a third circuit in a second circuitry at a different time. Additional and/or more specific examples of components with respect to machine 600 follow.

In some embodiments, machine 600 can operate as a standalone device or can be connected (e.g., networked) to other machines. In a networked deployment, machine 600 can operate in the capacity of a server machine, a client machine, or both in server-client network environments. In some examples, machine 600 can act as a peer machine in a peer-to-peer (P2P) (or other distributed) network environment. Machine 600 can be or include a PC, a tablet PC, a PDA, a mobile telephone, a web appliance, a network router, switch or bridge, an RFID smartcard or other proximity-based card, access control card, electronic key, key fob, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 can include a hardware processor 602 (e.g., a central processing unit (CPU), a graphics processing unit (GPU), a hardware processor core, or any combination thereof) and a main memory 604, a static memory (e.g., memory or storage for firmware, microcode, a basic-input-output (BIOS), unified extensible firmware interface (UEFI), etc.) 606, and/or mass storage 608 (e.g., hard drives, tape drives, flash storage, or other block devices) some or all of which can communicate with each other via an interlink (e.g., bus) 634. Machine 600 can further include a display device 610, an input device 612, and/or a user interface (UI) navigation device 614. Examples of suitable display devices include, without limitation, one or more LEDs, an LCD panel, a display screen, a touchscreen, one or more lights, etc. Example input devices and UI navigation devices include, without limitation, one or more buttons, a keyboard, a touch-sensitive surface, a stylus, a camera, a microphone, etc. In some examples, one or more of the display device 610, input device 612, and/or UI navigation device 614 can be a combined unit, such as a touch screen display. Machine 600 can additionally include a signal generation device 618 (e.g., a speaker), a network interface device 620, one or more antennas 630, a power source 632, and one or more sensors 616, such as a global positioning system (GPS) sensor, compass, accelerometer, or other sensor. Machine 600 can include an output controller 628, such as a serial (e.g., universal serial bus (USB)), parallel, or other wired or wireless (e.g., infrared (IR), NFC, etc.) connection to communicate with or control one or more peripheral devices (e.g., a printer, card reader, etc.).

Processor 602 can correspond to one or more computer processing devices or resources. For instance, processor 602 can be provided as silicon, as a Field Programmable Gate Array (FPGA), an Application-Specific Integrated Circuit (ASIC), any other type of Integrated Circuit (IC) chip, a collection of IC chips, or the like. As a more specific example, processor 602 can be provided as a microprocessor, Central Processing Unit (CPU), or plurality of microprocessors or CPUs that are configured to execute instructions sets stored in an internal memory 622 and/or memory 604, 606, or mass storage 608.

Any of memory 604, 606, or mass storage 608 can be used in connection with the execution of application programming or instructions by processor 602 for performing any of the functionality or methods described herein, and for the temporary or long-term storage of program instructions or instruction sets 624 and/or other data for performing any of the functionality or methods described herein. Any of memory 604, 606, or mass storage 608 can comprise a computer readable medium that can be any medium that can contain, store, communicate, or transport data, program code, or instructions 624 for use by or in connection with machine 600. The computer readable medium can be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or EEPROM), Dynamic RAM (DRAM), a solid-state storage device, in general, a compact disc read-only memory (CD-ROM), or other optical or magnetic storage device. As noted above, computer readable media includes, but is not to be confused with, computer readable storage medium, which is intended to cover all physical, non-transitory, or similar embodiments of computer readable media.

Network interface device 620 includes hardware to facilitate communications with other devices over a communication network, utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., 4G/5G cellular networks), Plain Old Telephone (POTS) networks, wireless data networks (e.g., IEEE 802.11 family of standards known as Wi-Fi), networks based on the IEEE 802.15.4 family of standards, and peer-to-peer (P2P) networks, among others. In some examples, network interface device 620 can include an Ethernet port or other physical jack, a Wi-Fi card, a Network Interface Card (NIC), a cellular interface (e.g., antenna, filters, and associated circuitry), or the like. In some examples, network interface device 620 can include one or more antennas to wirelessly communicate using, for example, at least one of single-input multiple-output (SIMO), multiple-input multiple-output (MIMO), or multiple-input single-output (MISO) techniques.

Antenna 630 can correspond to one or multiple antennas and can be configured to provide for wireless communications between machine 600 and another device. Antenna(s) 630 can be arranged to operate using one or more wireless communication protocols and operating frequencies including, but not limited to, the IEEE 802.15.1, Bluetooth, Bluetooth Low Energy (BLE), near field communications (NFC), ZigBee, GSM, CDMA, Wi-Fi, RF, UWB, and the like. By way of example, antenna(s) 630 can be RF antenna(s), and as such, may transmit/receive RF signals through free-space to be received/transferred by another device having an RF transceiver.

Power source 632 can be any suitable internal power source, such as a battery, capacitive power source or similar type of charge-storage device, etc., and/or can include one or more power conversion circuits suitable to convert external power into suitable power (e.g., conversion of externally-supplied AC power into DC power) for components of the machine 600. Power source 632 can also include some implementation of surge protection circuitry to protect the components of machine 600 from power surges.

As indicated above, machine 600 can include one or more interlinks or buses 634 operable to transmit communications between the various hardware components of the machine. A system bus 634 can be any of several types of commercially available bus structures or bus architectures.

The above detailed description includes references to the accompanying drawings, which form a part of the detailed description. The drawings show, by way of illustration, specific embodiments that can be practiced. These embodiments may also be referred to herein as “examples.” Such embodiments or examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. That is, the above-described embodiments or examples or one or more aspects, features, or elements thereof can be used in combination with each other.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure or portions thereof may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. In the context of this disclosure, a computer readable medium may be any medium that can contain, store, communicate, or transport the program for use by or in connection with the systems disclosed herein. As indicated above, the computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical, magnetic, or solid state storage device. As noted above, computer-readable media includes, but is not to be confused with, computer-readable storage medium, which is intended to cover all physical, non-transitory, or similar embodiments of computer-readable media.

In the foregoing description various embodiments of the present disclosure have been presented for the purpose of illustration and description. They are not intended to be exhaustive or to limit the invention to the precise form disclosed. Accordingly, other modifications or variations are possible in light of the above teachings.

What is claimed is:

1. A method for making a bioinformatics database of age-related macular degeneration (AMD) data, the method comprising:
obtaining live patient image data, the live patient image data including a first set of fundus photos from one or more live patient eyes;
obtaining eye bank image data, the eye bank image data including a second set of fundus photos captured from one or more postmortem eyes;
performing data analysis to correlate one or more characteristics of the live patient image data with one or more characteristics of the eye bank image data, wherein the correlated characteristics include one or more common image data characteristics of each image data set based on a disease progression of AMD; and
storing the common image data characteristics in a bioinformatics database.

2. The method of claim 1, further comprising:
obtaining tissue data, the tissue data including one or more tissue data characteristics produced from one or more biological analysis techniques performed on tissue samples from the postmortem eyes;
performing additional data analysis to correlate the tissue data characteristics with the common image data characteristics based on the disease progression of AMD; and
storing the tissue data characteristics in the bioinformatics database, wherein the tissue data characteristics are stratified by disease severity of AMD.

3. The method of claim 2, further comprising:
obtaining live patient observation data, the live patient observation data including one or more attributes of respective human subjects corresponding to the live patient eyes;
obtaining eye bank observation data, the eye bank observation data including attributes of respective human subjects corresponding to the postmortem eyes and measurements performed on the postmortem eyes;
performing additional data analysis to correlate clinical data characteristics from the live patient observation data with the eye bank observation data and with the common image data characteristics based on the disease progression of AMD; and
storing the clinical data characteristics in the bioinformatics database.

4. The method of claim 3, wherein the bioinformatics database establishes relationships among one or more attributes associated with the common image data characteristics, the clinical data characteristics, and the tissue data characteristics.

5. The method of claim 4, further comprising:
retrieving one or more of the common image data characteristics, the tissue data characteristics, or the clinical data characteristics from the bioinformatics database; and
generating a visual representation of one or more of the common image data characteristics, the clinical data characteristics, or the tissue data characteristics.

6. The method of claim 2, further comprising:
using the bioinformatics database to identify a therapeutic target in the tissue data;
wherein identification of the therapeutic target is performed using one or more of:
proteomic data analysis, transcriptomic data analysis, genomic data analysis, gene expression profiling, RNA or DNA sequencing, RNA or DNA methylation analysis, epigenetic modification analysis, post-translational proteomic modifications, metabolomic biomarker identification, structural biological identification, or therapeutic targeting signaling analysis.

7. The method of claim 1, further comprising:
retrieving the common image data characteristics from the bioinformatics database; and
providing the common image data characteristics for use in therapeutic target identification, the therapeutic target identification to be performed by one or more hypothesis generation function or computational biology function implemented in a computing system.

8. The method of claim 1, further comprising:
processing the first set and the second set of fundus photos with one or more artificial intelligence (AI) model, wherein the AI model is configured to perform a grading or classification on the first set or the second set of fundus photos to determine the disease progression of AMD.

9. The method of claim 8, wherein the first set and the second set of fundus photos provide stereoscopic color fundus images, and wherein each image of the first set and the second set of fundus photos is classified to a level of a multi-step grading system of an AMD disease stage.

10. The method of claim 1, wherein the data analysis to correlate the characteristics of the live patient image data with the characteristics of the eye bank image data includes using one or more artificial intelligence (AI) model to identify the common image data characteristics.

11. A non-transitory computer readable medium comprising executable software instructions, that when executed by one or more processor of a computing system, causes the computing system to perform operations including:
obtaining live patient image data, the live patient image data including a first set of fundus photos from one or more live patient eyes;
obtaining eye bank image data, the eye bank image data including a second set of fundus photos captured from one or more postmortem eyes;
performing data analysis to correlate one or more characteristics of the live patient image data with one or more characteristics of the eye bank image data, wherein the correlated characteristics include one or more common image data characteristics of each image data set based on a disease progression of AMD; and
storing the common image data characteristics in a bioinformatics database.

12. The non-transitory computer readable medium of claim 11, the operations further including:
obtaining tissue data, the tissue data including tissue data characteristics produced from biological analysis techniques performed on tissue samples from the postmortem eyes;
performing additional data analysis to correlate the tissue data characteristics with the common image data characteristics based on the disease progression of AMD; and
storing the tissue data characteristics in the bioinformatics database, wherein the tissue data characteristics are stratified by disease severity of AMD.

13. The non-transitory computer readable medium of claim 12, the operations further including:
obtaining live patient observation data, the live patient observation data including attributes of respective human subjects corresponding to the live patient eyes;
obtaining eye bank observation data, the eye bank observation data including attributes of respective human subjects corresponding to the postmortem eyes and measurements performed on the postmortem eyes;
performing additional data analysis to correlate clinical data characteristics from the live patient observation data with the eye bank observation data and with the common image data characteristics based on the disease progression of AMD; and
storing the clinical data characteristics in the bioinformatics database.

14. The non-transitory computer readable medium of claim 13, wherein the bioinformatics database establishes relationships among one or more attributes associated with the common image data characteristics, the clinical data characteristics, and the tissue data characteristics.

15. The non-transitory computer readable medium of claim 14, the operations further including:
retrieving one or more of the common image data characteristics, the tissue data characteristics, or the clinical data characteristics from the bioinformatics database; and
generating a visual representation of one or more of the common image data characteristics, the clinical data characteristics, or the tissue data characteristics.

16. The non-transitory computer readable medium of claim 12, the operations further including:
using the bioinformatics database to identify a therapeutic target in the tissue data;
wherein identification of the therapeutic target is performed using one or more of:
proteomic data analysis, transcriptomic data analysis, genomic data analysis, gene expression profiling, RNA or DNA sequencing, RNA or DNA methylation analysis, epigenetic modification analysis, post-translational proteomic modifications, metabolomic biomarker identification, structural biological identification, or therapeutic targeting signaling analysis.

17. The non-transitory computer readable medium of claim 11, the operations further including:
retrieving the common image data characteristics from the bioinformatics database; and
providing the common image data characteristics for use in therapeutic target identification, the therapeutic target identification to be performed by one or more hypothesis generation function or computational biology function implemented in a computing system.

18. The non-transitory computer readable medium of claim 11, the operations further including:
processing the first set and the second set of fundus photos with one or more artificial intelligence (AI) model, wherein the AI model is configured to perform a grading or classification on the first set or the second set of fundus photos to determine the disease progression of AMD.

19. The non-transitory computer readable medium of claim 18, wherein the first set and the second set of fundus photos provide stereoscopic color fundus images, and wherein each image of the first set and the second set of fundus photos is classified to a level of a multi-step grading system of an AMD disease stage.

20. The non-transitory computer readable medium of claim 11, wherein the data analysis to correlate the characteristics of the live patient image data with the characteristics of the eye bank image data includes using one or more artificial intelligence (AI) model to identify the common image data characteristics.

21. A method for using a bioinformatics database of age-related macular degeneration (AMD) data, the method comprising:
retrieving one or more common image data characteristics from a bioinformatics database, wherein the bioinformatics database correlates one or more characteristics of images from one or more live patients with one or more characteristics of images from one or more postmortem eyes based on a disease progression of AMD;
retrieving one or more tissue data characteristics from the bioinformatics database, wherein the tissue data characteristics are produced from one or more biological analysis techniques performed on tissue samples from the postmortem eyes, and wherein the bioinformatics database correlates the tissue data characteristics with the common image data characteristics based on the disease progression of AMD; and
identifying a therapeutic target based on the tissue data characteristics, wherein identification of the therapeutic target is performed using one or more of: proteomic data analysis, transcriptomic data analysis, genomic data analysis, gene expression profiling, RNA or DNA sequencing, RNA or DNA methylation analysis, epigenetic modification analysis, post-translational proteomic modifications, metabolomic biomarker identification, structural biological identification, or therapeutic targeting signaling analysis.
